SEQUENCE LISTING (30 sequences)

SEQ ID NO: 1 - RNA, 71 nt, E. coli
auucugugaggcccccauaaucuuucuccgucccgccaaguucgucgaaagauuguggggguuuuccuuuu

SEQ ID NO: 2 - RNA, 72 nt, E. coli
auucugugaggcccccacuaucuuucgucguuccgccaaguucguaagaaagauaguggggguuuuccuuuu

SEQ ID NO: 3 - RNA, 72 nt, Klebsiella pneumoniae
auucugugaacgccccuacuaucuuucacgagcccgccaaaguucgaggaaagauaguggggguuuuccuuu

SEQ ID NO: 4 - RNA, 70 nt, Escherichia coli
auucugugaggcccccauuauuuuucugcguuccgccaaguucgaggaaaaauaguggggguuuuccuuu

SEQ ID NO: 5 - RNA, 70 nt, Escherichia coli; identical to SEQ ID NO: 4

SEQ ID NO: 6 - RNA, 72 nt, Salmonella enterica; identical to SEQ ID NO: 2

SEQ ID NO: 7 - RNA, 72 nt, Salmonella typhimurium; identical to SEQ ID NO: 2

SEQ ID NO: 8 - DNA, 71 bp, E. coli
aaaaggaaaacccccacaatctttcgacgaacttggcgggacggagaaagattatgggggcctcacagaat

SEQ ID NO: 9 - DNA, 72 bp, E. coli
aaaaggaaaacccccactatctttcttacgaacttggcggaacgacgaaagatagtgggggcctcacagaat

SEQ ID NO: 10 - DNA, 72 bp, Klebsiella pneumoniae
aaaggaaaacccccactatctttcctcgaactttggcgggctcgtgaaagatagtaggggcgttcacagaat

SEQ ID NO: 11 - DNA, 70 bp, E. coli
aaaggaaaacccccactatttttcctcgaacttggcggaacgcagaaaaataatgggggcctcacagaat

SEQ ID NO: 12 - DNA, 70 bp, E. coli; identical to SEQ ID NO: 11

SEQ ID NO: 13 - DNA, 72 bp, Salmonella enterica; identical to SEQ ID NO: 9

SEQ ID NO: 14 - DNA, 72 bp, Salmonella typhimurium; identical to SEQ ID NO: 9

SEQ ID NO: 15 - DNA, 110 bp, E. coli
gggttgaatctctggtacggtttcattatactttacccgtattctgtgaggcccccataatctttctccgtcccgccaagttcgtcgaaagattgtgggggttttccttt

SEQ ID NO: 16 - DNA, 56 bp, artificial
  synthetic
agcttgggttgaatctctggtacggtttcattatactttacccgtattctgtgagg

SEQ ID NO: 17 - DNA, 60 bp, artificial
  synthetic
gtttccttttgggggtgttagaaagctgcttgaaccgccctgcctctttctaataccccc

SEQ ID NO: 18 - DNA, 49 bp, artificial
  synthetic
ctagcaaaggaaaacccccacaatctttcgacgaacttggcgggacgga

SEQ ID NO: 19 - DNA, 67 bp, artificial
  synthetic
acccaacttagagaccatgccaaagtaatatgaaatgggcataagacactccgggggtattagaaag

SEQ ID NO: 20 - DNA, 24 bp, artificial
  synthetic primer
ggctcttgtatctatcagtgaagc

SEQ ID NO: 21 - DNA, 19 bp, artificial
  synthetic primer
ctggagaggacgaatgacg

SEQ ID NO: 22 - DNA, 19 bp, artificial
  sythetic primer
cgcaccattatatctttgg

SEQ ID NO: 23 - DNA, 20 bp, artificial
  synthetic primer
gaccggttatcaccattccg

SEQ ID NO: 24 - DNA, 20 bp, artificial
  synthetic primer
ccgttggtgtagcttgctgc

SEQ ID NO: 25 - DNA, 21 bp, artificial
  synthetic primer
taagcgacagcttgtggcagg

SEQ ID NO: 26 - DNA, 20 bp, artificial
  sythetic primer
ggaatggtgctgtctagtgc

SEQ ID NO: 27 - DNA, 20 bp, artificial
  synthetic primer
cacttgcaggcttgaacagc

SEQ ID NO: 28 - DNA, 20 bp, artificial
  synthetic primer
tgctgttatcaacctcaccc

SEQ ID NO: 29 - DNA, 20 bp, artificial
  synthetic primer
ggaacaacggatcaacagcg

SEQ ID NO: 30 - DNA, 19 bp, artificial
  synthetic primer
atgacagcaggaatgtgcc

(19) United States
(12) Patent Application Publication
(10) Pub. No.: US 2008/0194032 A1
(43) Pub. Date: Aug. 14, 2008

(54) Methods and compositions for curing persistent I-complex super-family plasmids

(76) Inventors: Anthony A. Gatenby, Wilmington, DE (US); Monica Marie Olson, Newark, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(21) Appl. No.: 11/452,577

(22) Filed: Jun. 14, 2006

(51) Int. Cl. C12N 15/74

(52) U.S. Cl. 435/488

(57) ABSTRACT

Methods are provided for curing a host cell of a persistent plasmid of the I-complex super-family. The method proceeds via the direct introduction of a second plasmid containing a gene expressing at least a portion of an I-complex super-family-type Inc RNA. The second plasmid may be cured from the host cell, such as with the use of a temperature sensitive origin of replication.

### FIELD OF THE INVENTION

The present disclosure is in the field of microbial engineering. Specifically, it relates to a method and materials for eliminating an endogenous I-complex super-family plasmid from an E. coli production strain.

### BACKGROUND OF THE INVENTION

Strains of bacteria often harbor endogenous plasmids which are undesirable, particularly when they are present in bacterial hosts used in commercial production. These endogenous plasmids may affect the growth and/or the productivity of the bacterial strain. In addition, an endogenous plasmid may harbor a gene or multiple genes which encode proteins that are potentially toxic, and therefore may create a hazard. It is beneficial to eliminate such plasmids in hosts, especially in hosts used in a large-scale production situation.

Through sequencing of a derivative of E. coli strain ATCC13821, a 103 kb closed contiguous DNA sequence was identified that represented a plasmid, designated pMT100. The origin of replication region of pMT100 was identified by similarity to a homologous region in plasmid Collb-P9 (Hama, et al., Bact. 172: 1983-1991 (1990)). Sequence analysis indicated that this endogenous plasmid belongs to the IncI gamma incompatibility group of the I-complex super-family. The I-complex super-family includes plasmids of incompatibility groups B, Iγ, Iα, K, and Z (Praszkier and Pittard, Plasmid 53: 97-112 (2005)). Sequence analysis also indicated that the endogenous plasmid carried several genes encoding fimbrial protein subunits, which were identified as K88 antigen-specific polypeptides. The K88 antigen is known to be expressed on the surface of enterotoxigenic E. coli strains that cause illness and death in young pigs, due to binding of the antigen to the intestinal tract. Therefore it is desirable to eliminate this plasmid from strain ATCC13821, and any derivatives, as a step in the development of these strains as commercial production hosts.

Elimination of the endogenous plasmid was attempted by standard DNA intercalation methods, which were unsuccessful. High concentrations (10-fold above standard) of intercalating agents were then used, with risk of producing many genomic mutations, but still the plasmid was not successfully eliminated. The stability of the plasmid to elimination and its presence in the bacterial host over many generations without selection indicated that it is a particularly persistent plasmid. Another means is needed for eliminating the IncI persistent plasmid.

Incompatibility of plasmids has been used for displacing endogenous plasmids. Plasmids are classified into incompatibility groups, based on the inability of plasmids to coexist in a cell. Introduction into a cell of a plasmid which is naturally incompatible with an endogenous plasmid, can result in displacing the endogenous plasmid with the introduced plasmid.

The Collb-P9 plasmid has been studied as a member of the IncI alpha incompatibility group of the I-complex super-family (Asano and Mizobuchi, Journal of Biol. Chem. 273: 11815-11825 (1998); Asano et al., Journal of Biol. Chem. 274: 17924-17933 (1999); Asano and Mizobuchi, Journal of Biol. Chem. 275:1269-1274 (2000)). It was shown that the inc gene of the low copy number Coll-P9 plasmid encodes an antisense Inc RNA which is involved in regulating replication of the plasmid. The Inc RNA inhibits repY translation by binding to the repZ mRNA in the vicinity of the repY start codon in a region called loop I. RepY is a short open reading frame upstream of the repZ coding region, which encodes the replication leader peptide. Translation of repY induces formation of a pseudoknot between the region preceding the repZ start codon and loop I, allowing translation of repZ. The binding of Inc RNA with loop I also directly inhibits pseudoknot formation, and thereby inhibits repZ translation. This replication control system maintains a low copy number of the IncI alpha Collb-P9 plasmid in host cells. Replication of plasmids in the I-complex super-family is controlled by the same general mechanism, although plasmids belonging to different I-complex super-family incompatibility groups may replicate in the same cell (they are compatible).

US 2004/0224340 discloses a non-harmful conjugative displacing plasmid used for displacing a harmful plasmid in a bacterial cell. In this disclosure, an origin of transfer is present on a non-harmful plasmid, such that it is conjugatively transferred into a cell harboring a harmful plasmid, thereby eliminating the harmful plasmid and leaving the non-harmful plasmid in the cell.

There remains a need for a means of eliminating a persistent plasmid of the I-complex super-family, in which mutations are not generated, the persistent plasmid is rapidly lost, and the remaining host cell is plasmid-free.

### SUMMARY OF THE INVENTION

The invention relates to methods for curing plasmids of the I-complex super-family from host cells harboring the same. Accordingly the invention provides a method for curing a host cell of a persistent plasmid of the I-complex super-family comprising the steps of:
a) providing a host cell harboring a first plasmid of the I-complex super-family;
b) introducing into said host cell a second nonconjugative plasmid comprising an I-complex super-family Inc RNA encoding sequence and a marker to generate a transformed host cell;
c) growing said transformed host cell of (b) for at least one generation making use of the marker whereby the first plasmid is cured from a host cell having the marker.

In an alternative embodiment the invention provides a method for curing a host cell of a persistent plasmid of the I-complex super-family comprising the steps of:
a) providing a host cell harboring a first plasmid of the I-complex super-family;
b) introducing into said host cell a second plasmid comprising;
   i) an I-complex super-family-type Inc RNA encoding sequence to generate a transformed host cell;
   ii) a marker; and
   iii) a negative selection marker;
c) growing said transformed host cell of (b) for at least one generation under selective conditions whereby the first plasmid is cured from the host cell; and
d) growing said transformed host cell of (b) under the negative selection condition corresponding to the negative selection marker of (iii) whereby the second plasmid is cured from the host cell.

In another embodiment the invention provides a curing plasmid useful for the elimination of plasmids of the I-complex super-family comprising:

a) a negative selection marker

b) an I-complex super-family-type Inc RNA encoding sequence;

c) a marker.

### BRIEF DESCRIPTION OF THE SEQUENCE DESCRIPTIONS

The following sequences comply with 37 C.F.R. 1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (1998) and the sequence listing requirements of the EPC and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

A Sequence Listing is provided herewith on Compact Disk. The contents of the Compact Disk containing the Sequence Listing are hereby incorporated by reference in compliance with 37 CFR 1.52(e). The Compact Discs are submitted in duplicate and are identical to one another. The discs are labeled “Copy 1—Sequence Listing” and “Copy 2—Sequence listing” The discs contain the following file: Seq listing CL2989 final.ST25 having the following size: 7,000 bytes and which was created Jun. 14, 2006.

SEQ ID NO:1 is the sequence of the pMT100 Inc RNA.

SEQ ID NO:2 is the sequence of the Collb-P9 plasmid Inc RNA.

SEQ ID NO:3 is the sequence of the pIE545 (no3) Inc RNA.

SEQ ID NO:4 is the sequence of the pMU720 Inc RNA.

SEQ ID NO:5 is the sequence of the pMU707 Inc RNA.

SEQ ID NO:6 is the sequence of the pNF1358 Inc RNA.

SEQ ID NO:7 is the sequence of the R64 plasmid Inc RNA.

SEQ ID NO:8 is the sequence of pMT100 encoding Inc RNA.

SEQ ID NO:9 is the sequence of Collb-P9 encoding Inc RNA.

SEQ ID NO:10 is the sequence of pIE545 (no3) encoding Inc RNA.

SEQ ID NO: 11 is the sequence of pMU720 encoding Inc RNA.

SEQ ID NO:12 is the sequence of pMU707 encoding Inc RNA.

SEQ ID NO:13 is the sequence of pNF1358 encoding Inc RNA.

SEQ ID NO:14 is the sequence of R64 plasmid encoding Inc RNA.

SEQ ID NO:15 is the sequence of the pMT100 inc gene.

SEQ ID NOs:16-19 are the sequences of oligos used to make a pMT100 inc gene DNA fragment.

SEQ ID NO:20 is the sequence of the JW168-1 primer.

SEQ ID NOs:21-22 are the sequences of primers for the pilK gene.

SEQ ID NOs:23-34 are the sequences of primers for the abi gene.

SEQ ID NOs:25-26 are the sequences of primers for the repZ gene.

SEQ ID NOs:27-28 are the sequences of primers for the endo gene.

SEQ ID NOs:29-30 are the sequences of primers for the cscR gene.

### DETAILED DESCRIPTION OF THE INVENTION

The present invention describes a method of eliminating an endogenous persistent plasmid of the I-complex super-family from a host bacterial cell, which leaves the host cell plasmid-free. This method is useful in the construction of host strains to be used in the production of commercial products, where it is undesirable for the host cells to harbor endogenous, potentially detrimental plasmids.

The following definitions and abbreviations are to be use for the interpretation of the claims and the specification.

“Polymerase chain reaction” is abbreviated PCR.

“Carbenicillin” is abbreviated carb.

“Ampicillin” is abbreviated amp.

The term “persistent plasmid” means a plasmid that is highly stable in that it remains in a host over many generations without selection pressure, and is not cured from a host cell using standard curing methods.

The term “I-complex super-family-type” means derived from a plasmid that is a member of the I-complex super-family. A derived sequence may be identical to that found in the plasmid from which it came, which is the original sequence. In addition, a derived sequence may have some changes in the sequence whereby it retains the original function. Generally, a derived sequence will have about 87% identity to the original sequence. More suitable is a derived sequence having at least 95% identity to the original sequence, and most suitable is a derived sequence having at least 98% identity to the original sequence. The derived sequence may refer to either a sequence encoding an RNA, or to a sequence of an RNA.

The term “percent identity”, as known in the art, is a relationship between two or more polynucleotide sequences or two or more polypeptide sequences, as determined by comparing the sequences. In the art, “identity” also means the degree of sequence relatedness between polynucleotide or polypeptide sequences, as the case may be, as determined by the match between strings of such sequences. “Identity” and “similarity” can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignment of the sequences may be performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method are KTUPLE 1, GAP PENALTY=3, WINDOW=5 and DIAGONALS SAVED=5.

The term “marker” means a gene that confers a phenotypic trait that is easily detectable through screening or selection. A selectable marker is one wherein cells having the marker gene can be distinguished based on growth. For example, an antibiotic resistance marker serves as a useful selectable marker, since it enables detection of cells which are resistant to the antibiotic, when cells are grown on media containing that particular antibiotic. A marker used in screening is, for example, one whose conferred trait can be visualized. Genes involved in carotenoid production or that encode proteins (i.e. beta-galactosidase, beta-glucuronidase) that convert a colorless compound into a colored compound are examples of this type of marker. A screening marker gene may also be referred to as a reporter gene.

The term “making use of the marker” means identifying cells based on the phenotypic trait provided by the marker. The marker may provide a trait for identifying cells by methods including selection and screening.

The term “negative selection marker” means a DNA sequence which confers a property that is detrimental under particular conditions. The property may be detrimental to a plasmid or to a whole cell. To make use of the negative selection marker, cells are grown under the negative selection condition corresponding to the negative selection marker. For example, expression of a sacB gene in the presence of sucrose is lethal to the expressing cells. Another example is a temperature sensitive origin of replication, which is nonfunctional at non-permissive temperature such that the plasmid cannot replicate.

A nucleic acid fragment is “hybridizable” to another nucleic acid fragment, such as a cDNA, genomic DNA, or RNA, when a single stranded form of the nucleic acid fragment can anneal to the other nucleic acid fragment under natural conditions in a cell or in vitro under appropriate conditions of temperature and solution ionic strength. Hybridization and washing conditions are well known and exemplified in Sambrook, J., Fritsch, E. F. and Maniatis, T. Molecular Cloning: A Laboratory Manual, 2^{nd} ed., Cold Spring Harbor Laboratory: Cold Spring Harbor (1989), particularly Chapter 11 and Table 11.1 therein (entirely incorporated herein by reference).

The term “complementary” is used to describe the relationship between nucleotide bases that are capable of hybridizing to one another. For example, with respect to DNA, adenosine is complementary to thymine and cytosine is complementary to guanine. In hybridized DNA-RNA molecules, adenosine is complementary to uracil.

“Gene” refers to a nucleic acid fragment that expresses a specific functional RNA (such as an antisense RNA) or protein. It may or may not include regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature.

“Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing sites, effector binding sites and stem-loop structures.

“Transcriptional and translational control sequences” are DNA regulatory sequences, such as promoters, enhancers, terminators, and the like, that provide for the expression of a coding sequence in a host cell. In eukaryotic cells, polyadenylation signals are control sequences.

“Coding sequence” or “coding region” refers to a DNA sequence that codes for a specific amino acid sequence or that is transcribed to produce a functional RNA.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is recognized that since in most cases the exact boundaries of regulatory sequences have not been completely defined, DNA fragments of different lengths may have identical promoter activity.

The “3′ non-coding sequences” or “termination sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., the coding sequence is under the transcriptional control of the promoter). Coding sequences can be operably linked to regulatory sequences in sense or antisense orientation.

Standard recombinant DNA and molecular cloning techniques used here are well known in the art and are described by Sambrook, J., Fritsch, E. F. and Maniatis, T., Molecular Cloning: A Laboratory Manual, 2^{nd} ed., Cold Spring Harbor Laboratory: Cold Spring Harbor, N.Y. (1989) (hereinafter “Maniatis”); and by Silhavy, T. J., Bennan, M. L. and Enquist, L. W., Experiments with Gene Fusions, Cold Spring Harbor Laboratory: Cold Spring Harbor, N.Y. (1984); and by Ausubel, F. M. et al., Current Protocols in Molecular Biology, published by Greene Publishing Assoc. and Wiley-Interscience, Hoboken, N.J. (1987).

### Second Plasmid for Displacement of I-Complex Super-Family Plasmid Inc RNA and Encoding Sequence

Plasmids of the I-complex super-family express Inc RNA from an inc gene present on the plasmid. In the instant invention, Inc RNA is expressed from a plasmid that does not belong to the I-complex super-family so that it is able to replicate in a host harboring an I-complex super-family plasmid. The plasmid from which the Inc RNA is expressed is not able to undergo conjugation. It was found that with introduction of a non-conjugative, non-I-complex super-family plasmid that expresses I-complex super-family Inc RNA (herein called a second plasmid) into a host cell having a persistent plasmid of the I-complex super-family (herein called a first plasmid), the second plasmid can displace the first plasmid. Thus it was found that expression of Inc RNA in trans (on the second plasmid) did not only lower the copy number of the first plasmid, but that replication of the first plasmid was sufficiently blocked to cause elimination of the first plasmid from the host cell.

Inc RNA from any member of the I-complex super-family, including plasmids of incompatibility groups B, Iγ (I gamma), Iα (I alpha), K, and Z, may be used in the instant invention. In one aspect of the present method, the Inc RNA of the target first plasmid to be cured is expressed from the second plasmid. In another aspect of the present method, Inc RNA of a plasmid other than the target first plasmid to be cured is expressed from the second plasmid, with the Inc RNA expressed from the second plasmid having at least about 87% sequence identity with the Inc RNA sequence of the target plasmid. In this second aspect of the method, the curing is carried out at reduced temperature. Typically room temperature of about 21° C.-23° C. is used. It is thought that with the reduced temperature, the energy needed to maintain the hybridized RNA/RNA inhibition structure is less, so that the Inc RNA does not need to be exactly complementary the target binding RNA sequence for replication to be inhibited.

Examples of I-complex super-family RNAs include that encoded in the pMT100 plasmid (IncI gamma group; SEQ ID NO:1), that encoded in the plasmid IncI gamma minireplicon, which is identical to SEQ ID NO:1 (Accession # M20412; Nikoletti et al., J. Bacteriol. 170 (3), 1311-1318 (1988)), that encoded in the Collb-P9 plasmid (IncI alpha group; SEQ ID NO:2), that encoded in the pIE545 (no3) plasmid from Klebsiella pneumoniae (Inc Z group; Praszkier et al. (1991) J. Bact. 173(7): 2393-2397; SEQ ID NO:3), that encoded in the plasmid pMU720 minireplicon (IncB group; SEQ ID NO:4), that encoded in the pMU707 plasmid (SEQ D NO:5), and those encoded in plasmids pNF1358 and R64 from Salmonella (SEQ ID NOs:6 and 7). Any of these, or other Inc RNA sequences with identities of at least about 87% may be paired as the Inc sequence for expression from the second plasmid and the Inc sequence of the target first plasmid to be cured. For example, pIE546 IncZ RNA was expressed from a second plasmid and successfully cured the pMT100 plasmid when the present method was performed at room temperature. The IncZ and pMT100 Inc RNAs have 87.7% sequence identity.

The entire Inc RNA is typically 70 to 72 nucleotides in length. In the instant method, the expressed Inc RNA may be the entire naturally encoded sequence, or a portion of the natural Inc RNA sequence that is functional in the replication control system of an I-complex super-family plasmid. Thus the expressed Inc RNA may be an Inc RNA derived sequence. The Inc RNA derived sequence may have base changes from the natural Inc RNA sequence while maintaining the replication control function. Such a derived sequence will have about 87% identity to the original sequence. More suitable is a derived sequence having about 95% identity to the original sequence, and most suitable is a derived sequence having about 98% identity to the original sequence.

In the instant invention, the I-complex super-family Inc RNA is expressed from the sequence encoding that Inc RNA which is derived from a plasmid belonging to the I-complex super-family, specifically derived from the inc gene. Sequences encoding Inc RNAs that may be used to express the Inc RNAs include those of pMT100 (SEQ ID NO: 8), Collb-P9 plasmid (SEQ ID NO:9; GenBank # NC_{—}002122), pIE545 (no3) plasmid (SEQ ID NO:10; GenBank # M93064), plasmid pMU720 minireplicon (SEQ ID NO:11; GenBank # M28718), pMU707 plasmid (SEQ ID NO:12), and plasmids pNF1358 and R64 from Salmonella (SEQ ID NOs:13 and 14; GenBank #s DQ017661 and NC_{—}005014). The sequences given for the Inc RNAs and their encoding regions are useful in the present method, though there may be some variation in the exact 5′ and/or 3′ ends of the Inc RNAs and their encoding sequences since the ends have not been experimentally determined in all cases. I-complex super-family Inc RNA encoding sequence may refer to the sequence encoding an entire I-complex super-family Inc RNA, which may be used in the instant method. In addition, a sequence that encodes a portion of an Inc RNA that is able to bind (through RNA-RNA hybridization) to the repZ mRNA expressed from an I-complex super-family plasmid, including the loop I region, and inhibit repY translation and the pseudoknot formation required for repZ translation may be used and is referred to also as an 1-complex super-family-type Inc RNA encoding sequence. Base mismatches may be present between the repZ mRNA sequence and the expressed Inc RNA sequence, that do not affect the interaction of these two RNAs in regards to the replication control system. Thus the Inc RNA encoding sequence may be an inc gene derived sequence. The inc gene derived sequence may have base changes from the natural inc gene sequence while maintaining the replication control function. Such a derived sequence will have about 87% identity to the original sequence. More suitable is a derived sequence having about 95% identity to the original sequence, and most suitable is a derived sequence having about 98% identity to the original sequence. One of skill in the art will be able to readily determine the amount of mismatch allowed while retaining effectiveness for inhibition of I-complex super-family plasmid replication.

### Inc Gene

The Inc RNA may be expressed from the natural pMT100 inc gene (SEQ ID NO:8), or from an inc gene of another plasmid member of the I-complex super-family. In addition, a non-natural or chimeric gene may be used to express Inc RNA. Suitable regulatory sequences for expression of the Inc RNA coding region may be incorporated into a chimeric gene. Regulatory sequences include promoters, and terminators for transcription. The region encoding the Inc RNA may be expressed from an operably linked heterologous promoter that is active in the host cell harboring a persistent I-complex super-family plasmid. To achieve inhibition of replication of the first plasmid, expression from a heterologous promoter is at least as high as expression from the natural inc gene promoter. Many promoters known in the art to be useful for expression of chimeric genes may be used in the instant invention, including for example, lac, ara, tet, trp, lambdaIP_{L}, lambdaP_{R}, T7, T5, tac, trc, malE (maltose binding protein promoter) and derivatives thereof.

### Plasmid Marker

Typically, the plasmid which expresses the Inc RNA (second plasmid) also carries a marker. The marker provides a trait for identifying cells by methods including selection and screening. The marker is used to identify those cells that receive the second plasmid, which are the cells that potentially will have the first I-complex super-family plasmid displaced. Types of usable markers include screening and selection markers. Many different selection markers available for bacterial cell selection may be used, including nutritional markers, antibiotic resistance markers, metabolic markers, and heavy metal tolerance markers. Some specific examples include, but are not limited to, thyA, serA, ampicillin resistance, kanamycin resistance, carbenicillin resistance, and mercury tolerance. In addition, a screenable marker may be used to identify cells that have received the Inc RNA expressing plasmid. Examples of screenable markers include GFP, GUS, carotenoid production genes, and beta-galactosidase. A particularly suitable marker in the instant invention is a selectable marker.

Hosts and Introduction of Second Plasmid into Host Cells

The instant method may be used with any host cell that harbors a persistent plasmid of the I-complex super-family. I-complex super-family plasmids will typically be found in bacterial cells, such as Escherichia, Salmonella, Klebsiella, Pseudomonas, and Citrobacter. It is particularly useful to eliminate I-complex super-family persistent plasmids from E. coli cells that are used as hosts for genetic engineering for commercial purposes.

The nonconjugative second plasmid may be introduced into cells having an I-complex super-family plasmid by any direct method. Such methods are well known in the art and may include uptake in calcium treated cells, electroporation, and freeze-thaw uptake.

### Plasmid Displacement

In the instant invention, cells are grown for multiple generations, making use of the second plasmid marker to identify and propagate cells having the second plasmid. Cells may be identified using a screening marker. More suitably, cells having resistance to the selection corresponding to a resistance marker are grown under selective conditions for multiple generations. Cells are then tested for loss of the I-complex super-family plasmid. Typically PCR, using primers derived from the I-complex super-family plasmid, is used to detect loss of the first plasmid. Other plasmid analysis methods may be used such as restriction digestion and gel electrophoresis. The first persistent plasmid is in the majority of cases lost after growth. Typically the persistent plasmid is lost after growth for at least about 10 generations. Growth for at least about 5 generations may also be effective. In a rare cell, the persistent plasmid may remain. When expressing an Inc RNA that is other than identical to the sequence of the Inc RNA of the first plasmid, more generations of growth may be used. For sequences of about 87% identity, up to about 80 generations of growth may be used. A cell with a remaining persistent plasmid is easily detected using standard molecular techniques known to one skilled in the art, and may be avoided in further experimentation.

### Plasmid-Free Host

The Inc RNA may be expressed from any plasmid that is able to replicate in the host cell of the target I-complex super-family first plasmid, that is not able to undergo conjugation, and that can be cured from the host cell. Plasmids that are easily eliminated by standard treatment with DNA intercalation agents, or other standard plasmid elimination methods known to one skilled in the art may be used. A plasmid having a negative selection marker may be used, where growth under the negative selection conditions corresponding to the negative selection marker eliminates cells harboring the plasmid. Remaining cells will have lost the plasmid. Particularly useful are plasmids having a temperature sensitive origin of replication, such as pJW168 (Wild et al., Gene 223: 55-66 (1998)). This type of plasmid may be readily eliminated from a host cell by growing the cell at a restrictive temperature for plasmid replication. For example, a typical temperature sensitive origin of replication functions at an incubation temperature of 30° C., while at 42° C., replication is defective and the plasmid is lost from the host cell.

### EXAMPLES

The present invention is further defined in the following Examples. It should be understood that these Examples are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations used is as follows: “min” means minute(s), “h” means hour(s), “μL” means microliter(s), “mL” means milliliter(s), “L” means liter(s), “nm” means nanometer(s), “mm” means millimeter(s), “cm” means centimeter(s), “μm” means micrometer(s), “mM” means millimolar, “M” means molar, “mmol” means millimole(s), “μmole” means micromole(s), “g” means gram(s), “μg” means microgram(s), “ng” means nanogram(s), “mg” means milligram(s), “g” means the gravitation constant, “rpm” means revolutions per minute,

### General Methods

Standard recombinant DNA and molecular cloning techniques used in the Examples are well known in the art and are described by Sambrook, J., Fritsch, E. F. and Maniatis, T., Molecular Cloning: A Laboratory Manual, Cold Spring Harbor Laboratory Press, Cold Spring Harbor, N.Y., 1989, by T. J. Silhavy, M. L. Bennan, and L. W. Enquist, Experiments with Gene Fusions, Cold Spring Harbor Laboratory, Cold Spring Harbor, N.Y., 1984, and by Ausubel, F. M. et al., Current Protocols in Molecular Biology, Greene Publishing Assoc. and Wiley-Interscience, N.Y., 1987.

Materials and methods suitable for the maintenance and growth of bacterial cultures are also well known in the art. Techniques suitable for use in the following Examples may be found in Manual of Methods for General Bacteriology, Phillipp Gerhardt, R. G. E. Murray, Ralph N. Costilow, Eugene W. Nester, Willis A. Wood, Noel R. Krieg and G. Briggs Phillips, eds., American Society for Microbiology, Washington, D.C., 1994, or by Thomas D. Brock in Biotechnology: A Textbook of Industrial Microbiology, Second Edition, Sinauer Associates, Inc., Sunderland, Mass., 1989. All reagents, restriction enzymes and materials used for the growth and maintenance of bacterial cells were obtained from Aldrich Chemicals (Milwaukee, Wis.), BD Diagnostic Systems (Sparks, Md.), Life Technologies (Rockville, Md.), DIFCO Laboratories (Detroit, Mich.), or Sigma Chemical Company (St. Louis, Mo.), unless otherwise specified.

### Growth Conditions and Materials

Typically, bacteria were incubated in LB medium in 15-ml snap cap tubes or 125 ml flasks. For maintenance of plasmid pInc1, strains were grown at 30° C. or 37° C. During the pInc1 curing process, strains were incubated at 42° C.

### Electroporation

Electrocompetent bacterial cells and DNA were mixed together and transferred to a 2 mm gap cuvette (BTX, San Diego, Calif.) on ice. After 5 minutes the cuvette was placed into the slider connected to a BTX ECM600 electroporator. The settings were as follows: resistance of 129 ohms, charging voltage of 2.45 kV, and pulse length of 5-6 msec. Immediately after delivery of the electrical pulse, 800 μl of SOC media was added and the cuvette contents were transferred to a 15 ml tube. The tube was incubated at 30° C. (37° C. for DH5α), shaking at 225 rpm, for 1 hour, then 10 μl, 50 μl, and 200 μl aliquots of the culture were spread onto LB agar plates containing 100 μg/ml ampicillin or carbenicillin. The plates were incubated 16-24 hours, until individual colonies were approximately 2 mm in diameter.

### PCR Conditions

Unless specifically noted, all PCR reactions were carried out using the HotStarTaq Master Mix Kit (Qiagen; Valencia, Calif.). The Master Mix contained: 10 units/μl HotStarTaq DNA polymerase, PCR buffer with 3 mM MgCl₂, and 400 μM each dNTP. Reactions were carried out in 15 μl volumes in either 96-well plates or 0.2 ml eppendorf tubes. Five to ten pmoles of each primer were present, as well as either a small amount of cell material or 250 picograms of genomic DNA, prepared with the Puregene kit from Gentra Systems (Minneapolis, Minn.) as template. Samples were heated in a Mastercycler gradient thermocycler (Eppendorf, Westbury, N.Y.) to 95° C. for 15 min in order to activate the polymerase, then 30 cycles followed consisting of: 94° C. for 30 sec, 55° C. or 58° C. for 30 sec, and 72° C. for 1 min. Finally, the samples were kept at 72° C. for 10 min for final polymerization. The tubes or plate were then cooled to 4° C. until removed from the thermocycler.

### Example 1

### Cloning the Inc Gene

The sequence of the region of pMT100 containing the inc gene (SEQ ID NO:15) was used to design oligonucleotides which could be joined to form the inc gene. Four synthetic oligonucleotides (SEQ ID NO:16 to SEQ ID NO:19) were obtained that, when annealed together, produce a double stranded DNA fragment, each strand being 116 b long. This DNA fragment includes the inc gene promoter, region encoding the Inc RNA, and inc gene terminator. At the 5′ end of the annealed dsDNA was a 5′ overhang that is compatible with HindIII-digested dsDNA. At the 3′ end of the annealed fragment was a 5′ overhang that is compatible with NheI-digested dsDNA. To a single tube was added: 500 pmoles of each oligonucleotide (20 μl total), 25 μl nuclease-free water, and 5 μl 10× adaptor buffer (250 mM Tris-HCl (pH8), 100 mM MgCl₂). The mixture was heated to 95° C. for 5 minutes, then allowed to cool slowly to room temperature over 1.5 hours. The resulting fragment was named inc, and it was calculated to be at a concentration of 30.6 ng/μl.

Two hundred ng of pJW168 (Wild et al., Gene 223: 55-66 (1998)) was digested with HindIII and NheI. The pJW168 plasmid carries an amp resistance gene, a carbenicillin resistance gene, and a temperature sensitive origin of replication. The reaction was electrophoresed on an agarose/TBE gel containing ethidium bromide, and the DNA visualized using long wave UV radiation. The 6 kb fragment representing the linearized plasmid was excised from the gel, purified with the Zymoclean gel DNA recovery kit (Zymo Research, Orange, Calif.), and ligated to 5 μl of the inc DNA fragment. A fifth of the ligation reaction was electroporated into DH5α cells as described in the General Methods. Transformants were selected on LB+amp (100 μg/ml) plates. Restriction digests performed on plasmid DNA extracted from selected colonies indicated the presence of the desired construct. The inc DNA fragment insert in the plasmid was sequenced with primer JW168-1 (SEQ ID NO:20), confirming the correct insert and its orientation. The plasmid was named pInc1.

### Example 2

### Curing of pMT100 Using pInc1

E. coli ATCC13821 was grown during the day to an OD₆₀₀ Of approximately 1.0. Three mls of the culture were centrifuged, and the cell pellet was washed twice in ice cold autoclaved water: the first time with 1.5 ml, the second with 700 μl. After each resuspension the mixture was centrifuged and the supernatant discarded. The final pellet was resuspended in 50 μl of autoclaved water. The prepared cells were electroporated with 200 ng of pInc1. Transformants were selected on LB+amp (100 μg/ml) plates. Individual colonies were inoculated into 3 mls of LB+carbenicillin (100 μg/ml) liquid medium and incubated at 37° C. with shaking at 225 rpm. Carbinicillin selection gives cleaner transformation plates with fewer satellite colonies. After 10 generations of growth a 1×10⁻⁶ dilution was made in LB from each culture and 50 to 100 μl aliquots were spread onto LB+carbenicillin (100 μg/ml) plates. Colony PCR was performed in 96 well microtiter plates on individual colonies that arose after 24 hours of incubation. In the first screening, primers for either pilK (SEQ ID NO:21 and SEQ ID NO:22) or abi (SEQ ID NO:23 and SEQ ID NO:24) were used. Both genes are located on pMT100, so any clones not producing a 562 bp (pilK) or 626 bp (abi) PCR product were negative for the plasmid. All but one of 48 colonies tested were pMT100 negative.

Two clones that passed the first screening were inoculated into liquid LB+carb medium and grown overnight. Genomic DNA was isolated from 0.5 ml of each culture with the Puregene kit (Gentra Systems, Minneapolis, Minn.). PCR amplifications were performed using these genomic DNAs and also on genomic DNA from a plasmid bearing strain, DPD4145, that was descended from ATCC13281. The primers used in the first two sets of reactions were again for pilK and abi. In the other sets were primers for repZ (SEQ ID NO:25 and SEQ ID NO:26), endo (SEQ ID NO:27 and SEQ ID NO:28), and cscR (SEQ ID NO:29 and SEQ ID NO:30). RepZ is a gene on pMT100 that is essential for its replication, while endo and cscR are chromosomal genes. The pMT100-cured strains, collectively named DPD6026, did not amplify pilK, abi, or repZ (1058 bp), whereas these products were amplified using DNA prepared from DPD4145. All strains produced endo (955 bp) and cscR (1428 bp) fragments, which proved that nothing in the genomic DNAs inhibited the PCR reactions, and that these were ATCC13281-derived strains.

### Example 3

### Curing pInc1

E. coli DPD6026 was streaked onto an LB plate. The plate was incubated overnight at 42° C., then a single colony was picked and streaked onto a fresh LB plate. The incubation and restreaking was repeated once more. Colonies from the third plate were streaked onto both LB and LB+amp plates and incubated at 37° C. overnight. All grew on the LB plate, but most streakings on the LB+amp plate did not grow. One of these was selected as a plasmidless strain named DPD6027. A plasmid DNA miniprep performed on a culture of DPD6027 with the Wizard Plus SV Minipreps DNA Purification System (Promega, Madison, Wis.) confirmed the absence of plasmid, as none could be visualized from 10 μl of miniprep DNA electrophoresed on an agarose/TBE gel.

### Example 4

### Curing of pMT100 Using Overexpression of IncZ at Reduced Temperature

Plasmid pMT100 belongs to the I-complex super-family, which includes plasmids of incompatibility groups B, Iγ, Iα, K, and Z (Praszkier and Pittard, Plasmid 53: 97-112 (2005)). This example was done to determine whether pMT100 could be cured by expression of an inc sequence derived from a plasmid of another incompatibility group of the I-complex super-family. A reduced temperature was used to allow pairing of sequences with some mismatch.

The published sequence of the incZ gene (SEQ ID NO:19) was used to design oligonucleotides which could be joined to recreate the gene. Four synthetic oligonucleotides (SEQ ID NO:4, SEQ ID NO:20, SEQ ID NO:21, and SEQ ID NO:22) were obtained that, when annealed together, produce a double stranded DNA fragment, each strand being 118 nucleotides long. This DNA fragment includes the inc gene promoter, region encoding the IncZ RNA, and incZ gene terminator. At the 5′ end of the annealed dsDNA was a 5′ overhang that is compatible with HindIII-digested dsDNA. At the 3′ end of the annealed fragment was a 5′ overhang that is compatible with NheI-digested dsDNA. To a single tube was added: 500 pmoles of each oligonucleotide (20 μl total), 25 μl nuclease-free water, and 5 μl 10× adaptor buffer (250 mM Tris-HCl (pH8), 100 mM MgCl₂). The mixture was heated to 95° C. for 5 minutes, then allowed to cool slowly to room temperature over 1.5 hours. The resulting fragment was named incZ, and it was calculated to be at a concentration of 30.6 ng/μl.

Two hundred ng of pJW168 (Wild et al., Gene 223: 55-66 (1998)) was digested with HindIII and NheI. The pJW168 plasmid carries an amp resistance gene, a carbenicillin resistance gene, and a temperature sensitive origin of replication. The reaction was electrophoresed on an agarose/TBE gel containing ethidium bromide, and the DNA visualized using long wave UV radiation. The 6 kb fragment representing the linearized plasmid was excised from the gel, purified with the Zymoclean gel DNA recovery kit (Zymo Research, Orange, Calif.), and ligated to 5 μl of the incZ DNA fragment. A fifth of the ligation reaction was electroporated into DH5α cells as described in the General Methods. Transformants were selected on LB+amp (100 μg/ml) plates. Restriction digests performed on plasmid DNA extracted from selected colonies indicated the presence of the desired construct. The incZ DNA fragment insert in the plasmid was sequenced with primer JW168-1 (SEQ ID NO:20), confirming the correct insert and its orientation. The plasmid was named pInc3Z.

E. coli ATCC13821 was grown during the day to an OD600 of approximately 1.0. Three mls of the culture were centrifuged, and the cell pellet was washed twice in ice cold autoclaved water: the first time with 1.5 ml, the second with 700 μl. After each resuspension the mixture was centrifuged and the supernatant discarded. The final pellet was resuspended in 50 μl of autoclaved water. The prepared cells were electroporated with 200 ng of pInc3Z. Transformants were selected on LB+amp (100 μg/ml) plates. Individual colonies were inoculated into 3 mls of LB+carbenicillin (100 μg/ml) liquid medium and incubated at about 21° C. with shaking at 225 rpm. After approximately 80 generations of growth, a 1×10⁻⁶ dilution was made in LB from each culture and 50 to 100 μl aliquots were spread onto LB+carbenicillin (100 μg/ml) plates. Colony PCR was performed on individual colonies that arose after 24 hours of incubation. In the first screening, primers for pilK (SEQ ID NO:21 and SEQ ID NO:22) were used. The gene was located on pMT100, so any clones not producing a 562 bp PCR product were negative for the plasmid. One of the 24 clones tested produced such a negative result. Five more colony PCR reactions were performed to verify the absence of the plasmid and to rule out the possibility of a contaminant. The primers used in the first reaction were again for pilK. In the other four were primers for abi (SEQ ID NO:23 and SEQ ID NO:24), repZ (SEQ ID NO:25 and SEQ ID NO:26), endo (SEQ ID NO:27 and SEQ ID NO:28), and cscR (SEQ ID NO:29 and SEQ ID NO:30). The pMT100-cured strain, named DPD6026.5, did not amplify plasmid genes pilK, abi, or repZ (1058 bp), but did amplify chromosomal genes endo (955 bp) and cscR (1428 bp) fragments, which proved that pMT100 was indeed missing and that this was an ATCC13281-derived strain.

What is claimed is:

1. A method for curing a host cell of a persistent plasmid of the I-complex super-family comprising the steps of:
a) providing a host cell harboring a first plasmid of the I-complex super-family;
b) introducing into said host cell a second nonconjugative plasmid comprising an I-complex super-family Inc RNA encoding sequence and a marker to generate a transformed host cell;
c) growing said transformed host cell of (b) for at least one generation making use of the marker whereby the first plasmid is cured from a host cell having the marker.

2. A method according to claim 1 wherein the growing of the host cell in step (c) is for at least about 5 generations.

3. A method according to claim 1 wherein the I-complex super-family-type Inc RNA encoding sequence is at least 87% identical to the Inc RNA encoding sequence of the pMT100 plasmid (SEQ ID NO:8).

4. A method according to claim 1 wherein the second plasmid is introduced into the host cell according to a direct DNA uptake method.

5. A method according to claim 1 wherein the second plasmid comprises a negative selection marker.

6. A method according to claim 5 wherein the negative selection marker comprises a temperature sensitive origin of replication.

7. A method according to claim 6 wherein the second plasmid is cured from the host cell by growing the cell at non-permissive temperature.

8. A method according to claim 1 wherein the host cell is a bacteria.

9. A method according to claim 8 wherein the host cell is selected from the group consisting of Escherichia, Salmonella, Klebsiella, Pseudomonas, and Citrobacter.

10. A method according to claim 1 wherein the first persistent plasmid carries a gene encoding a toxic moiety.

11. A method according to claim 10 wherein the toxic moiety is selected from the group consisting K88 antigen-specific polypeptides.

12. A method for curing a host cell of a persistent plasmid of the I-complex super-family comprising the steps of:
a) providing a host cell harboring a first plasmid of the I-complex super-family;
b) introducing into said host cell a second plasmid comprising;
i) an I-complex super-family-type Inc RNA encoding sequence to generate a transformed host cell;
ii) a marker; and
iii) a negative selection marker;
c) growing said transformed host cell of (b) for at least one generation under selective conditions whereby the first plasmid is cured from the host cell; and
d) growing said transformed host cell of (b) under the negative selection condition corresponding to the negative selection marker of (iii) whereby the second plasmid is cured from the host cell.

13. A method according to claim 12 wherein the negative selection marker is a temperature sensitive origin of replication and the restrictive condition is restrictive temperature.

14. A curing plasmid useful for the elimination of plasmids of the I-complex super-family comprising:
a) a negative selection marker
b) an I-complex super-family-type Inc RNA encoding sequence;
c) a marker.
